SEQUENCE LISTING (12 sequences)

SEQ ID NO: 1 - protein, 22 aa, SARS-COV-2
SFIEDLLFNKVTLADAGFIKQY

SEQ ID NO: 2 - protein, 22 aa, SARS-COV-1
SFIEDLLFNKVTLADAGFMKQY

SEQ ID NO: 3 - protein, 21 aa, SARS-COV-2
KQYGDCLGDIAARDLICAQKF

SEQ ID NO: 4 - protein, 21 aa, SARS-COV-1
KQYGECLGDINARDLICAQKF

SEQ ID NO: 5 - protein, 7 aa, SARS-COV-2
GDIAARD

SEQ ID NO: 6 - protein, 7 aa, Rubella virus
GDDSGRD

SEQ ID NO: 7 - protein, 7 aa, SARS-COV-2
KQYGDCL

SEQ ID NO: 8 - protein, 6 aa, HCoV-229E
KRYGFC

SEQ ID NO: 9 - protein, 18 aa, SARS-COV-2
SFIEDLLFNKVTLADAGF

SEQ ID NO: 10 - protein, 4 aa, SARS-COV-2
KQYG

SEQ ID NO: 11 - protein, 5 aa, SARS-COV-2
CLGDI

SEQ ID NO: 12 - protein, 10 aa, SARS-COV-2
ARDLICAQKF

(19) United States
(12) Patent Application Publication
(10) Pub. No.: US 2026/0207540 A1
(43) Pub. Date: Jul. 23, 2026

(54) VIRUS TREATMENT METHODS, AND RELATED PHARMACEUTICAL COMPOSITIONS, VACCINE COMPOSITIONS AND SANITIZING COMPOSITIONS

(71) Applicant: Daniel Patrick Cashman, Albany, CA (US)

(72) Inventor: Daniel Patrick Cashman, Albany, CA (US)

(21) Appl. No.: 17/916,558

(22) PCT Filed: Mar. 31, 2021
(86) PCT No.: PCT/US2021/025122

§ 371 Date: Sep. 30, 2022

Related U.S. Application Data
(60) Provisional application No. 63/003,007, filed on Mar. 31, 2020.
(60) Provisional application No. 63/033,274, filed on Jun. 2, 2020.

(51) Int. Cl. A61K 31/198; A61K 9/00; A61K 31/137; A61K 39/00; A61K 39/215; A61P 31/14

(52) U.S. Cl. CPC A61K 31/198; A61K 9/0078; A61K 31/137; A61K 39/215; A61P 31/14; A61K 2039/5254

(57) ABSTRACT

Pharmaceutical compositions comprising at least one calcium chelating agent such as disodium ethylenediamine tetraacetate (Na2EDTA) as an active pharmaceutical ingredient, are described that are useful for treating an infection by single stranded RNA virus, such as SARS-COV-2, are described. Development of the compositions was assisted by using a novel drug discovery method which utilizes the characterization of a common molecular mechanism in a cohort of an unforeseen clinical co-morbidly patterns identified as outliers. Reverse engineering of the outlier cohort yielded unrecognized calcium requirements for infection by SARS-CoV-2 provided a common molecular mechanism in the outlier group that enabled formulation of the new pharmaceutical compositions.

### CROSS-REFERENCE TO RELATED APPLICATIONS

The present application, Appl. Ser. No. 17/916,558, filed Sep. 30, 2022, is a national phase application pursuant to 35 USC § 371 that claims priority from International Appl. Ser. No. PCT/US2021/025122, filed Mar. 31, 2021, now expired, which claims priority to U.S. provisional application Ser. No. 63/003,007, filed Mar. 31, 2020, now abandoned, and U.S. provisional application Ser. No. 63/033,274, filed Jun. 2, 2020, now abandoned, the disclosures of which are incorporated by reference herein in their entireties.

### SEQUENCE LISTINGS

Incorporated by reference herein is a sequence listing in ASCII text file format submitted via the USPTO patent electronic filing system complying with WIPO Standard ST.25 requirements having the file name of 17_916558_DC_AC_001US_ST25.txt, file size 2615 bytes, created on 2023 Nov. 20 using the Patent-in version 3.5 software.

### FIELD OF THE INVENTION

This patent specification relates to the field of coronavirus treatment and prevention methods, pharmaceutical, vaccine, and sanitizing compositions for said methods, and drug discovery methods for developing said compositions. More specifically, this patent specification relates to those treatment and prevention methods, pharmaceutical, vaccine, and sanitizing compositions, and drug discovery methods related to the single stranded RNA viruses including SARS-CoV-2 virus, also known as COVID-19, SARS-CoV-1 virus, HCoV-229E virus, MERS virus, Rubella virus, mutated variants thereof and other viruses utilizing calcium dependent processes to infect and/or replicate.

### BACKGROUND OF THE INVENTION

Members of the coronavirus (CoV) family that normally infect wild animal can appear in new human hosts and cause unforeseen severe disease as exemplified by infections with SARS-CoV in 2003, MERS-CoV in 2012, and SARS-CoV-2/COVID-19 in 2020. There are few, if any, effective options for these emerging viral diseases. Generally, in the absence of effective treatments specific for those viral infections, treatments intended and approved for other diseases are administered to patients with emerging viral syndromes. Those treatments are empirically based on very limited clinical or laboratory data. Moreover, the few U.S. Food and Drug Administration (FDA) approved therapies that have been deployed for antiviral activity against coronavirus include lopinavir (LPV), ritonavir (RTV), interferon beta (IFNb) and chloroquine. However, those small molecule drugs were selected for current human Coronavirus therapies primarily based on an August 2014 in vitro study. (de Wilde A H, et al. “Screening of an FDA-approved compound library identifies four small-molecule inhibitors of Middle East respiratory syndrome coronavirus replication in cell culture” Antimicrob. Agents Chemother. 2014 August; 58(8):4875-84).

Approximately 7 months after the de Wilde et al. study was published, researchers published a new mechanism to explain the increased efficiency of some virus infections discovered in airborne Rubella virus, which is a single stranded RNA virus unrelated to the Coronavirus family. (Dubé M, et al. “Rubella virus: first calcium-requiring viral fusion protein” PLoS Pathog. 2014 Dec. 4; 10(12)). Importantly, Dubé et al demonstrated that addition of the calcium chelator, EDTA completely abrogated Rubella fusion in vitro. None of the small molecule drugs selected to date for the treatment of CoV infection target disrupting a calcium-dependent fusion process most likely because such a process was not known to be involved in these Coronavirus viral infections.

Since the drug development process from discovery of a new to approved drug generally takes over 10 years, it is unrealistic to expect development of a novel small molecule drug for SARS-CoV-2/COVID-19. The strategies for Coronavirus treatment regimens have mainly relied on combination therapies with drugs known to have acceptable safety profiles, which include IFNs, ribavirin, and corticosteroids. However, data from past regimens indicate such treatments are not effective in treating a SARS-CoV-1 infection and therefore would unlikely be effective in treating a viral infection from SARS-CoV-2. (Totura A L and Bavari S., “Broad-spectrum coronavirus antiviral drug discovery” Expert Opin. Drug Discov. 2019 April; 14(4):397-412). Moreover, no perceived benefit, and possible deleterious effects were observed when corticosteroids (methylprednisolone) were given as treatment during the SARS-CoV-1 and MERS epidemics (Stockman U, Bellamy R, and Garner P. “SARS: systematic review of treatment effects” PLoS Med. 2006; 3(9):e343; Morra M E, Van Thanh L, Kamel M G, et al. “Clinical outcomes of current medical approaches for Middle East respiratory syndrome: a systematic review and meta-analysis” Rev. Med. Virol. 2018; 28(3):e1977). Additionally, recent clinical commentary indicates that corticosteroids should not be given routinely for the treatment of COVID-19. Accordingly, the asthma paradox in which asthma is considered a high medical risk factor for susceptibility to SARS-CoV-2/COVID-19 infection, yet asthma is not on the list of top 10 chronic health problems suffered by people who died from SARS-CoV-2/COVID-19 is unlikely due to steroid treatments because recent admonitions against routine systematic corticosteroids for the treatment of COVID-19 and prior reports indicate that systemic steroids for treating SARS-CoV-1 actually may have been harmful. (Russell C D et al., “Clinical evidence does not support corticosteroid treatment for 2019-nCoV lung injury” Lancet 2020, Feb. 15; 395(10223):473-475).

There currently is no specific antiviral drug for treating or preventing an infection by a Coronavirus, such as SARS-CoV-1, HCoV-229E and SARS-CoV-2, also known as COVID-19, MERS, and mutated variants thereof. Although there now exists vaccines that are effective for preventing infections by SARS-CoV-2/COVID-19, it is unclear if treatments with those vaccines will also prevent transmission from a vaccinated, asymptomatic individual to one that is non-vaccinated. Furthermore, those vaccines appear to provide reduced protection from infections in varying degrees by current SARS-CoV-2/COVID-19 mutated variants and it is unclear what level of protection, if any, would be provided for future variants as the virus continues to mutate. Therefore, there exists the need for additional vaccines that are effective in reducing transmission of SARS-CoV-2/COVID-19 infections and that would also remain effective for treatment of viral infections from current and future Cov-2 mutated variants.

In addition, achieving herd immunity with the currently available vaccines will be hampered by vaccine hesitancy by some individual in the US and elsewhere due to the unfortunate politicization of public health in that country. Therefore, there exists a need for a small molecule therapy to treat and interrupt viral transmission from those individuals, irrespective of the mutation status of the virus. That is expected to have a positive worldwide effect on transmissibility as the virus does not respect country borders or cares about one's political affiliation.

It is also unclear as to the persistence of protection offered by current vaccines, which may require yearly vaccinations or the continued development of new vaccines to address emerging variants. Besides attenuating acute infection, suitable treatments for chronic infections, so called “long haulers” are also urgently needed. A small molecule that addresses the underlying cause common to Coronavirus infection as disclosed herein would provide a solution to that problem.

In view of the aforementioned problems to be solved and the absence of suitable animal models for testing potential therapies for preventing or treating Coronavirus infections, there exists a need to find a molecular explanation(s) common to those infections in order to assist in developing novel coronavirus treatment methods including those using small molecule pharmaceutical compositions and vaccine compositions as described herein.

### BRIEF SUMMARY OF THE INVENTION

Novel virus treatment methods, drug discovery methods, pharmaceutical compositions, and vaccine compositions for use against coronaviruses, including SARS-CoV-2, also known as COVID-19, SARS-CoV-1, HCoV-229E, MERS virus, and other single stranded RNA viruses that are now discovered to be dependent on calcium for infection, are provided.

In one principle embodiment a pharmaceutically acceptable formulation is provided, wherein the formulation is comprised or consists essentially of: a) calcium chelating agent, or pharmaceutically acceptable salt thereof, as an active pharmaceutical ingredient of the formulation; b) one or more pharmaceutically acceptable excipients; and c) optionally a beta-2 agonist as another active pharmaceutical ingredient of the formulation.

In another principle embodiments a method is a method of treating or preventing a viral infection in a subject is provided, wherein the viral infection is through fusion of a virus to cells of the subject by a calcium-dependent pathway, the method comprising the step of: administering to the subject a therapeutically effective amount of a pharmaceutically acceptable formulation comprised or consisting essentially of: a) calcium chelating agent, or pharmaceutically acceptable salt thereof, as an active pharmaceutical ingredient of the formulation; b) one or more pharmaceutically acceptable excipients; and c) optionally a beta-2 agonist as another active pharmaceutical ingredient of the formulation.

In yet another principle embodiment a vaccine composition is provided comprising a live, attenuated coronavirus comprising a SARS-CoV-2 variant in which the SARS-CoV-2 fusion loops (amino acids 816-855) are replaced with the less pathogenic HCoV-229E fusion loop (amino acids 923-982) to provide substantially the same antigenicity of SARS-CoV-2 while limiting its pathogenicity to no more than the level of HCoV-229E.

### BRIEF DESCRIPTION OF THE DRAWINGS

FIG. 1. Shows an example of the calcium targets where EDTA can disrupt the coronavirus fusion loop process sites of the virus spike protein, which are believed to be necessary for infection (Millet, J K and Whittaker, GR, Virology 2018 517: 3-8).

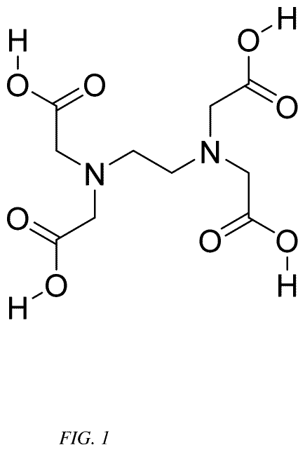

FIG. 2. Shows the structure of Disodium EDTA (Na₂EDTA) having the chemical formula of C₁₀ H₁₄ N₂ Na₂ O₈ and chemical identity CAS No. 139-33-3.

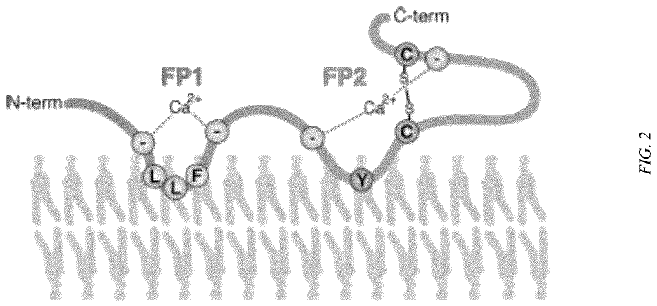

### DETAILED DESCRIPTION OF THE INVENTION

### Definitions

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof unless such presence or addition destroys the functionality of the invention or renders it unsuitable for its intended purpose. It will also be further understood that the terms “consisting essential of” and “consists essentially of” allows for the presence or additional features, steps, operations, elements, and/or components that do not materially affect the basic and novel characteristic(s) of the claimed invention. For example, allowing for the presence of an additional active pharmaceutical ingredient in a pharmaceutically acceptable formulation not related to the basic and novel characteristic(s) of the claimed formulation will have a material effect on that formulation.

Unless otherwise defined, all terms (including technical and scientific terms) used herein have the same meaning as commonly understood by one having ordinary skill in the art to whom the invention most closely pertains. It will be further understood that terms, such as those defined in commonly used medical dictionaries retain their meaning within the context of the relevant art and the present disclosure.

As used in this application, the term “about” or “approximately” refers to a range of values within plus or minus 10% of the specified number.

As used in this application, the term “substantially” means that the actual value is within about 10% of the actual desired value, typically within about 5% of the actual desired value and more typically within about 1% of the actual desired value of any variable, element or limit set forth herein.

As used in this application, the phrase “calcium-dependent virus fusion” is to be understood to at least include the virus infectious process whereby the peptide fusion loops located in the virus surface protein complex a metal ion between them by interactions to facilitate the insertion of the virus into the target host cell membrane, fusion.

As used in the application the term “chelating agent” refers a chemical compounds that is capable of reacting with metal ions to form a stable, water-soluble complex having a ring-like center with at least two bonds to the metal ion so as to inhibit or prevent it reacting as it would normally.

As used in this application the term “calcium chelating agent” is a chelating agent that is capable of reacting with calcium ions to form a stable, water-soluble complex having a ring-like center with at least two bonds to the calcium ion so as to inhibit or prevent it reacting as it would normally. In one aspect cleavage of a protein by a calcium-dependent protease is inhibited or prevented by the calcium chelating agent. In another aspect of the invention calcium-dependent fusion of a virus to host cells is inhibited or prevented by the calcium chelating agent. In the context of the application it is to be understood that a calcium chelating agent is an active pharmaceutical ingredient in a pharmaceutically acceptable formulation and not an excipient of the formulation.

A calcium chelating agent includes EDTA (ethylenediaminetriacetic acid), HEDTA (hydroxyethyl-ethylenediaminetriacetic acid) British Anti Lewisite (BAL), also known as 2,3-dimercaptopropanol, 2,3-dimercaptopropane 1-sulfoniv acid (DMPS), meso 2,3-dimercapto succinic acid (DMSA), other metal chelating agents useful for treating heavy metal poisoning, and pharmaceutically acceptable salts thereof and hydrates thereof, including hydrate forms of the pharmaceutically acceptable salts.

EDTA and HEDTA, which are encompassed by the term “calcium chelating agent”, in pharmaceutically acceptable salt forms include calcium disodium EDTA, diammonium EDTA, dipotassium EDTA, disodium EDTA, tetraethanolammonium-EDTA (TEA-EDTA), tetrasodium EDTA, tripotassium EDTA, trisodium EDTA trisodium HEDTA dinatrium ethylene diamine tetraacetate and further include hydrate forms thereof.

As used in this application, the term “Na₂EDTA” is understood to mean disodium ethylenediamine tetraacetate (disodium EDTA, also known as disodium edetate), which is an exemplary pharmaceutically acceptable salt of ethylenediamine tetraacetic acid (EDTA). EDTA, its salts and hydrates thereof (known collectively as edetates, unless explicitly stated otherwise or implied by context), are used in pharmaceutical products for treating heavy metal poisoning and have been approved as a food additive (E385). The molecular formula of disodium EDTA is C₁₀ H₁₄ N₂ Na₂ O₈ with a molecular weight of 336.21 and chemical identity CAS No. 139-33-3. The chemical identity of its dihydrate form having the molecular formula of C₁₀ H₁₄ N₂ Na₂ O₈·2H₂O is CAS No. 6381-92-6 with a molecular weight of 372.24.

As used in this application, the term “delivery method” is understood to mean a drug delivery system that is utilized to provide and maintain therapeutic concentrations of drug at the target biological site and includes diverse routes of administration such as oral ingestion, inhalation, and intravenous administration which may be utilized individually or in combination. For example, inhaled medications can be absorbed quickly and reach the lungs and upper respiratory track. Thus, inhalation of nebulized solutions can deliver Na₂EDTA or other calcium chelating agent directly into the very tissues typically attacked by SARS-CoV-2/COVID-19: lung alveoli, bronchi, larynx mouth nose, pharynx and throat.

As used in this application, the term “coronavirus” refers to a family of single stranded RNA viruses that cause a variety of respiratory, gastrointestinal, and neurological diseases in humans and other animals. While the Coronavirus family of single-stranded RNA viruses is divided into four genera: α-CoVs, β-CoVs, γ-CoVs, and δ-CoVs, only alpha and beta can infect mammals. (Yin Y and Wunderink R G, “MERS, SARS and other coronaviruses as causes of pneumonia”, Respirology_2018 Feb. 23(2):130-137).

After binding to their respective receptors, the Coronavirus viruses enter cells through endocytosis with the viral spike proteins driving the fusion of viral and endosome membranes to enable insertion of the viral genome into the cytoplasm. (Hoffmann M et al. “SARS-CoV-2 cell entry depends on ACE2 and TMPRSS2 and is blocked by a clinically-proven protease inhibitor” Cell 2020 Apr. 16; 181(2):271-280). The less pathogenic alpha-corona virus 229E (HCoV-229E) was isolated from students suffering from the common cold in 1966. (Hamre D, Procknow J J, “A new virus isolated from the human respiratory tract”, Proc. Soc. Exp. Biol. Med. 1966, January; 121(1):190-3; Almeida J D and Tyrrell D A. “The morphology of three previously uncharacterized human respiratory viruses that grow in organ culture”, J. Gen. Virol. 1967 April; 1(2):175-178).

HCoV-229E is highly prevalent and most people experience acute infection during their childhood. (Shirato K. et al., “Differences in neutralizing antigenicity between laboratory and clinical isolates of HCoV-229E isolated in Japan in 2004-2008 depend on the S1 region sequence of the spike protein”, J. Gen. Virol. 2012, 93:1908-191). One study found 650% of the children between the age of 2.5 and 3.5 years were seropositive for HCoV-229E. (Dijkman R et al. “Human coronavirus NL63 and 229E seroconversion in children” J. Clin. Microbiol. 2008, 46:2368-2373). The HCoV-229E virus binds to the aminopeptidase N receptor (CD13) (Yeager C L et al., “Human aminopeptidase N is a receptor for human coronavirus 229E”, Nature 1992, 357:420-2) and enters the cell after cleavage by TMPRSS2 and fusion. (Shirato K et al., “Clinical isolates of human coronavirus 229E bypass the endosome for cell entry”, J. Virol. 2017, 91:JVI.01387-16). The more pathogenic SARS-CoV-1 and SARS-CoV-2 (COVID-19) viruses belong to the β-genus.

The beta-corona virus SARS-CoV2 is a positive-sense single-stranded ribonucleic acid (ssRNA) of approximately 29700 nucleotides in length, of about 80% identical to that of SARS-CoV-1 and approximately 96% identical to the bat coronavirus BatCoV RaTG13. (Paraskevis D et al. “Full-genome evolutionary analysis of the novel corona virus (2019-nCoV) rejects the hypothesis of emergence as a result of a recent recombination event”, Infect. Genet. Evol. 2020 April, 79: 104212). The Spike (S) protein is 1273 amino acid long and S viral envelope protein that has two main subunits (S1 and S2) which protrude outwards with a ‘corona’ like appearance and binds to the angiotensin-converting enzyme 2 (ACE2) receptors. (Hoffmann M et al., “SARS-CoV-2 cell entry depends on ACE2 and TMPRSS2 and is blocked by a clinically proven protease inhibitor”, Cell 2020, doi.org/10.1016/j.cell. 2020.02.052; Walls A C et al. “Structure, function, and antigenicity of the SARS-CoV-2 spike glycoprotein” Cell 2020, doi.org/10.1016/j.cell.2020.02.058). The amino-terminal subunit is responsible for receptor binding and is labeled the S1 domain. The C-terminal part, labeled the S2 domain, contains the fusion machinery. More specifically, amino acids 318-510 of the S1 represent the receptor-binding domain (RBD) that binds to ACE2. (Wan Y et al. “Receptor recognition by the novel coronavirus from Wuhan: an analysis based on decade-long structural studies of SARS Coronavirus” J. Virol. 2020 Mar. 17, 94(7), e00127-20 doi: 10.1128/JVI.00127-20).

CoV S proteins have two cleavage sites and protease cleavage is required for S2 fusion to the cell membrane. There is an S1/S2 site composed of the amino acids RSVR that is located at the border between the S1 and S2 subunits and an S2′ site, composed of the amino acids RSAR. In SARS-CoV-2, the S2′ site is located at amino acid 815, just upstream of the putative fusion loop peptides present within the S2 subunit discussed below. In SARS-CoV-2/COVID-19 the type II transmembrane serine protease (TTSP) TMPRSS2 cleaves the S1-S2 subunits. (Coutard B et al., “The spike glycoprotein of the new coronavirus 2019-nCoV contains a furin-like cleavage site absent in CoV of the same clade”, Antiviral Res. 2020, 176:104742) doi: 10.1016/j.antiviral.2020.104742). It is also noteworthy that TMPRSS2 has two calcium-binding domains; a SRCR (scavenger receptor cysteine-rich) domain (aa 149-242) and a LDLRA (LDL receptor class A) domain (aa 113-148) that forms a binding site for calcium. (Paoloni-Giacobino A et al., “Cloning of the TMPRSS2 gene, which encodes a novel serine protease with transmembrane, LDLRA, and SRCR domains and maps to 21q22.3”, Genomics 1997 Sep. 15, 44(3): 309-20). The SRCR is a conserved calcium-dependent domain in which binding was disrupted by EDTA. (Reichhardt M P et al. “Structures of SALSA/DMBT1 SRCR domains reveal the conserved ligand-binding mechanism of the ancient SRCR fold”, Life Sci. Alliance, 2020 Feb. 25, 3(4) e201900502, doi: 10.26508/lsa.201900502). Together, the LDLRA and SRCR-like domains are believed to serve as substrate recognition sites.

New coronavirus treatment methods, drug discovery methods, pharmaceutical compositions, and vaccine compositions are discussed herein. In one aspect the present invention includes a novel therapy to treat or prevent a single stranded RNA virus infection, including a coronavirus virus infection in patients, such as SARS-CoV-2/COVID-19, based, in part, on novel protein sequence analyses of the virus's protein domains. Those analyses have identified loops in the spike protein receptor domains that require cleavage by host protease TMPRSS2 in order to initiate virus infectivity through fusion of the virus to host cells. Because the TMPRSS2 protease contains calcium-dependent domains for binding, it is predicted that decreasing free Ca²⁺ concentrations in the vicinity of tissues of a human subject that are infected or at risk of infection by a single stranded RNA virus would also reduce TMPRSS2 protease activity required for initiating fusion of the virus to host cells. Administration of a calcium-chelating agent would therefore treat and/or prevent infection by a single stranded RNA virus infection by interfering with that viral fusion.

In the following description, for purposes of explanation, numerous specific details are set forth in order to provide a thorough understanding of the present invention. It will be evident, however, that practicing the present invention by one of ordinary skill in the art is not limited to these specific details.

The present disclosure is to be considered as an exemplification of the invention and is not intended to limit the invention to the specific embodiments illustrated by the figures or description below.

The invention will now be further described by way of reference only to the following non-limiting examples. It should be understood, however, that the examples following are illustrative only, and should not be taken in any way as a restriction on the generality of the invention described herein.

### EMBODIMENTS

In some embodiments, a pharmaceutically acceptable formulation is provided wherein the formulation is comprised or consists essentially of at least one calcium chelating agent or a suitable pharmaceutically acceptable salt and/or hydrate form thereof; one or more suitable pharmaceutically acceptable excipients; and optionally a beta-2 agonist in effective amount to reduce bronchoconstriction. Preferably the at least one calcium chelating agent is disodium edetate, disodium edetate dihydrate or other edetate that contain EDTA in effective molar amount to treat or prevent a Coronavirus infection or other calcium-dependent viral infection.

In some embodiments, the pharmaceutically acceptable formulation is a pharmaceutically acceptable nebulizing formulation. In some embodiments the pharmaceutically acceptable nebulizing formulation is administered through a mechanical ventilator in conjunction with a beta-2 agonist, preferably a short acting beta-2 agonist, if not already present in the formulation, in order to reduce bronchoconstriction resulting from edetate inhalation. In some other embodiments, patients without access to a respirator are similarly treated with the pharmaceutically acceptable nebulizing formulation through a nebulizer facemask. Then, if tolerated, the more stable patients in some embodiments are subsequently treated as an outpatient using a nebulizer for inhalation delivery of a calcium chelating agent and beta-2 agonist solution, such as an EDTA/Albuterol solution wherein the solution is capable of nebulization.

In other embodiments a pharmaceutically acceptable intravenous formulation for treating or preventing a coronavirus infection is provided comprised or consisting essentially of at least one calcium chelating agent, or a suitable pharmaceutically acceptable salt and/or hydrate form thereof, and one or more suitable pharmaceutically acceptable excipients. Preferably at least one calcium chelating agent is an edetate such as Na₂EDTA·2H₂O.

In other embodiments a pharmaceutically acceptable oral formulation for treating or preventing a coronavirus infection, in particular a gastrointestinal coronavirus infection, or other single stranded RNA viral infection is provided comprised or consisting essentially of at least one calcium chelating agent, or a suitable pharmaceutically acceptable salt and/or hydrate form thereof and one or more suitable pharmaceutically acceptable excipients. Preferably at least one calcium chelating agent is an edetate such as Na₂EDTA·2H₂O.

In still other embodiments, prevention of a coronavirus or other calcium-dependent viral infection is achieved by adding at least one calcium chelating agent or a pharmaceutically acceptable salt and/or hydrate form thereof, such as Na₂EDTA·2H₂O, to soaps or alcohol-based hand sanitizers, lotions or sprays.

According to one group of embodiments consistent with the principles of the invention, pharmaceutical compositions for the treatment or prevention of a coronavirus infection such as those caused by SARS-CoV-2 virus, also known as the COVID-19 virus, SARS-CoV-1 virus, HCoV-229E virus, and MERS virus, or other calcium-dependent virus infections are provided.

In some embodiments, a pharmaceutically acceptable formulation for the treatment of a SARS-CoV-2 virus infection in a human subject is provided comprised or consisting essentially of at least one calcium chelating agent, or a pharmaceutically acceptable salt and/or hydrate form thereof, preferably an edetate such as Na₂EDTA·2H₂O, for providing EDTA in effective molar amount for disrupting a calcium-dependent pathway required for the infection.

The pharmaceutically acceptable formulations described herein are suitable for administration to a human subject in need thereof by any suitable delivery method wherein the at least one calcium chelating agent, or a pharmaceutically acceptable salt and/or hydrate form thereof, contains EDTA in effective molar amount for disrupting a calcium-dependent virus pathway responsible for the viral infection, wherein the calcium-dependent virus pathway is involved in fusion of the virus to cells of the human subject, also referred to as the host cells.

In any one of the embodiments described herein, the at least one calcium chelating agent in pharmaceutically acceptable salt and hydrate form is pharmaceutical grade disodium ethylenediamine tetraacetate dihydrate (Na₂EDTA·2H₂O).

Preferred delivery methods for administering a pharmaceutically acceptable formulations comprised or consisting essentially of a calcium chelating agent, or a pharmaceutically acceptable salt and/or hydrate form thereof, are by inhalation, oral ingestion, or intravenous injection, wherein the pharmaceutically acceptable formulation for delivery by intravenous injection or oral ingestion preferably omit the beta-2 agonist and wherein the pharmaceutically acceptable formulations for inhalation preferably include the beta-2 agonist.

In further embodiments, a pharmaceutically acceptable formulation is provided comprised or consisting essentially of:
a) at least one calcium chelating agent, or a pharmaceutically acceptable salt and/or hydrate form thereof, capable of disrupting a calcium-dependent protein cleavage pathway required for a viral infection, wherein said disrupting is by inhibition of a calcium-dependent host protease that cleaves a protein of a virus responsible for the viral infection. and
b) one or more pharmaceutically acceptable excipients, each of which is suitable for nebulization, wherein at least one of the pharmaceutically acceptable excipients is a liquid carrier, wherein the liquid carrier provides the pharmaceutically acceptable formulation as a pharmaceutically acceptable liquid formulation capable of nebulization; and.
c) optionally a beta-2 agonist,
wherein the calcium chelating agent in pharmaceutically acceptable salt and/or hydrate form is preferably an edetate such as Na₂EDTA·2H₂O; and/or
wherein the calcium-dependent host protease is preferably TMPRSS2.

In other embodiments a pharmaceutically acceptable formulation is provided comprised or consisting essentially of:
a) at least one calcium chelating agent, or a pharmaceutically acceptable salt and/or hydrate form thereof, capable of disrupting a calcium-dependent protein cleavage pathway required for a viral infection, wherein said disrupting is by inhibition of a calcium-dependent host protease that cleaves a protein of a virus responsible for the viral infection and
b) one or more pharmaceutically acceptable excipients, wherein at least one of the pharmaceutically acceptable excipients is a liquid carrier, wherein the liquid carrier provides the pharmaceutically acceptable formulation as a pharmaceutically acceptable liquid formulation for intravenous administration,
wherein the calcium chelating agent in pharmaceutically acceptable salt and/or hydrate form is preferably an edetate, such as Na₂EDTA·2H₂O; and/or
wherein the calcium-dependent host protease is preferably TMPRSS2.

In any one of the embodiments described herein, at least one of the pharmaceutically acceptable excipients is a pharmaceutically acceptable carrier. In some preferred embodiments the pharmaceutically acceptable carrier provides a liquid formulation suitable for intravenous injection. In other preferred embodiments the pharmaceutically acceptable carrier provides a formulation capable of nebulization for inhalation of the pharmaceutically acceptable formulation.

In any one of the embodiments described herein, at least one of the pharmaceutically acceptable excipients is a pharmaceutically acceptable carrier. In some preferred embodiments the pharmaceutically acceptable carrier provides a solid or liquid formulation suitable for oral administration or administration as a suppository, preferably in use for treating a gastrointestinal infection by a single stranded RNA viral infection, such an infection by a coronavirus.

In some preferred embodiments, the at least one calcium chelating agent of the pharmaceutically formulations is capable of interfering with fusion of a single stranded RNA virus, such as a coronavirus, with host cells.

In some other preferred embodiments, the at least one calcium chelating agent of the pharmaceutically formulations is capable of inhibiting cleavage by host protease TMPRSS2 of single stranded RNA virus protein, such as a coronavirus spike protein receptor domain to an extent that interferes with virus fusion with host cells.

In other preferred embodiments, pharmaceutically acceptable formulations are provided that decrease virus infection exposure wherein the pharmaceutically acceptable formulation is in the form of alcohol-based hand sanitizers, lotions, sprays or soaps. In some of those embodiments those pharmaceutically acceptable formulations are prepared by incorporating the calcium chelating agent into commercial alcohol-based hand sanitizers, lotions, sprays or soaps.

In some embodiment, the calcium chelating agent of the pharmaceutically acceptable formulation interferes with the calcium-dependent virus cleavage by host protease TMPRSS2 of a virus spike protein receptor domain required for viral fusion to host cells or interferes with protease cleavage of a viral protein domain(s) other than the virus spike protein receptor binding surface so as to decrease viral infectivity.

In another group of embodiments pharmaceutically acceptable formulations are provided comprised or consisting essentially of:
a) an inhibitor of host TMPRSS2, or other inhibitor of a protease responsible for cleavage of viral fusion peptides, also known as fusion loops, wherein the protease responsible for cleavage of viral fusion peptides contains calcium (II) metal binding sites,
b) one or more pharmaceutically acceptable excipients; and
c) optionally a beta-2 agonist.

Preferably, in those other embodiments the inhibitor of the host TMPRSS2 protease, or other calcium-dependent protease responsible for cleavage of viral fusion peptide(s) required for viral fusion to host cells, is a pharmaceutical grade edetate such disodium edetate or disodium ethylenediamine tetraacetate dihydrate

In further embodiments, a pharmaceutically acceptable formulation is provided comprised or consisting essentially of:
a) at least one calcium chelating agent, or a pharmaceutically acceptable salt thereof, wherein the calcium chelating agent, or it's pharmaceutically acceptable salt and/or hydrate form, wherein the salt and/or hydrate form contains an effective molar amount of the calcium chelating agent for inhibiting cleavage by host TMPRSS2 protease, or cleavage by other host protease(s), of a single stranded viral protein so as to disrupt calcium-dependent fusion of the virus to host cells,
b) one or more pharmaceutically acceptable excipients and
c) at least one beta-2 receptor agonist or one or more other pharmaceuticals useful in treating the symptom(s) of bronchoconstriction,
wherein the pharmaceutically acceptable formulation is in unit dosage form effective for treatment of a coronavirus infection.

In preferred embodiment at least one of the pharmaceutically acceptable excipients is a pharmaceutically acceptable carrier.

In preferred embodiments at least one of the pharmaceutically acceptable excipients is a pharmaceutically acceptable carrier, wherein the carrier in the unit dosage form is a diluent. In one such embodiment the diluent provides the pharmaceutically acceptable formulation in unit dosage form, wherein the pharmaceutically acceptable formulation is capable of nebulization for inhalation delivery of the formulation. In other preferred embodiments at least one of the pharmaceutically acceptable excipients is a pharmaceutically acceptable carrier, wherein the carrier in the unit dosage form is a diluent to provide a solution suitable for intravenous delivery of the unit dosage form.

In any one of the above embodiments a preferred pharmaceutically acceptable formulation is in the form of a nebulizer solution, wherein the unit dosage form of that solution contains an effective amount of the calcium chelating agent, or pharmaceutically acceptable salt and/or hydrate form thereof, or host TMPRSS2 protease inhibitor, optionally in pharmaceutically acceptable salt and/or hydrate form, in particular the unit dosage form contains between about 0.5 to about 10.0 mg/mL disodium edetate, preferably between about 0.7 to 2.0 mg/mL of disodium edetate, or other edetate salt and/or hydrate form containing an equimolar amount of EDTA. In any one of those preferred embodiments, the calcium chelating agent in pharmaceutically acceptable salt and hydrate form is preferably pharmaceutical grade disodium ethylenediamine tetraacetate dihydrate (Na₂EDTA·2H₂O). In any one of those nebulizer solutions, the beta-2 agonist is albuterol or metaproterenol or a pharmaceutically acceptable salt thereof in effective amount for alleviating a symptom from bronchoconstriction due to administration to a human subject of the EDTA unit dosage form by inhalation.

According to another group of embodiments consistent with the principles of the invention, a method of treatment is provided for a calcium-dependent viral infection. In some of those embodiments, a method for the treatment, amelioration or prevention of a viral infection caused by a Coronavirus, in particular a 0-corona virus, preferably a SARS-CoV-2 virus, is provided, the method comprising administering to a human subject in need thereof of a pharmaceutically acceptable formulation in unit dosage from consisting essentially of an effective amount of a calcium chelating agent, or pharmaceutically acceptable salt and/or hydrate form thereof, wherein the calcium chelating agent is capable of disrupting calcium-dependent virus protein cleavage by host protease TMPRSS2 or virus fusion to cells of the subject, (ii) one or more pharmaceutically acceptable excipients and (ii) an optionally a beta-2 receptor agonist, preferably one that is present in effective amount and is known to those skilled in the art for treating a human airway disease or to alleviate a symptom of bronchoconstriction from edetate inhalation. Preferably, the calcium chelating agent in pharmaceutically acceptable salt form is pharmaceutical grade calcium disodium ethylenediamine tetraacetate (Na₂EDTA) and in pharmaceutically acceptable salt form and hydrate form is pharmaceutical grade disodium ethylenediamine tetraacetate dihydrate.

In some of those embodiments, the method of treatment is by administrating the unit dosage form through an intravenous route, wherein the beta-2 receptor agonist is preferably omitted.

In some other of those embodiments, the method of treatment is by administrating the unit dosage form through an aerosol route by inhalation wherein the beta-2 receptor agonist is preferably present.

In some other of those embodiments, the method of treatment is by administrating the unit dosage form orally or rectally, wherein the beta-2 receptor agonist is preferably omitted. In some embodiments, a method is provided for the treatment, amelioration or prevention of a viral condition or disease caused by a Coronavirus, in particular a β-corona virus, preferably SARS-CoV-2 virus, the method comprising the steps of:
1) administering by aerosol to a human subject in need thereof of a pharmaceutically acceptable formulation in unit dosage from comprised or consisting essentially of an effective amount of a calcium chelating agent, or pharmaceutically acceptable salt and/or hydrate form thereof, wherein the calcium chelating agent is capable of disrupting calcium-dependent cleavage by host protease TMPRSS2 of a single stranded RNA virus protein or virus fusion of a single stranded RNA virus to cells of the subject, (ii) one or more pharmaceutically acceptable excipients, wherein at least one of the pharmaceutically acceptable excipients is a diluent to provide a solution suitable for said administrating.
2) co-administering a pharmaceutically acceptable formulation in unit dosage form comprised of a beta-2 receptor agonist, or pharmaceutically acceptable salt thereof, in an effective amount to prevent, treat or alleviate a symptom of bronchoconstriction and one or more pharmaceutically acceptable excipient, wherein said co-administration is per oral or wherein said administration is by an intravenous route and at least one of the pharmaceutically acceptable excipients is a diluent to provide a solution suitable for said intravenous administration.

Preferably, the calcium chelating agent in pharmaceutically acceptable salt form is an edetate salt, such as disodium edetate (Na₂EDTA), and preferably, the calcium chelating agent in pharmaceutically acceptable salt and hydrate form is pharmaceutical grade disodium ethylenediamine tetraacetate dihydrate (Na₂EDTA·2H₂O).

According to other embodiments consistent with the principles of the invention, vaccine compositions are provided which induce humeral immunity against SARS-CoV-2 coronavirus in a human subject. In some of those embodiments, a vaccine composition is comprised of a live, attenuated coronavirus comprising a variant in one or more of the amino acids in one or more of the calcium binding fusion loops of the virus spike protein in the region near amino acids 816 through 855 is mutated by genetic engineering, including replacing SARS-CoV-2 fusion loops (amino acids 816-855) with the less pathogenic HCoV-229E fusion loop (amino acids 923-982) to provide the antigenicity of COVID-19 while limiting the pathogenicity to the level of HCoV-229E The attenuated coronavirus is used in some embodiments for preparing the vaccine composition for treating and/or preventing a disease, such as infectious bronchitis from a single stranded RNA viral infection, e.g. COVID-19 infection, in a subject.

In some embodiments, a vaccine composition comprises a hybrid virus suitable for an attenuated alpha-beta SARS-CoV-2/COVID-19 vaccine is genetically engineered based upon the fusion loop homologies between the beta-corona SARS-CoV-2 virus and the less pathogenic alpha-corona HC0V-229E virus. Thus, replacing SARS-CoV-2 fusion loops (amino acids 816-855) with the less pathogenic HCoV-229E fusion loop (amino acids 923-982) is expected to provide antigenicity of COVID-19, but limit the pathogenicity to the level of HCoV-229E.

According to another aspect consistent with the principles of the invention, sanitizing compositions are provided for sanitizing objects and surfaces exposed or in contact with SARS-CoV-2 and other single stranded RNA virus are provided. In some embodiments, a sanitizing composition may comprise approximately at least 0.07% disodium edetate by weight, preferably in solution in an alcohol-based solvent, such as ethyl alcohol or isopropyl alcohol.

According to another aspect consistent with the principles of the invention, a drug discovery method is provided which may utilize the characterization of a molecular mechanism to explain unforeseen clinical co-morbidly patterns identified as outliers derived from understanding the molecular mechanism of the outlier to formulate a new therapeutic agent is provided. In some embodiments, the drug discovery method may include the steps of: 1) identifying outliers of unexpected disease pattern(s) in the co-morbidity data reflecting the expected disease prevalence for a matched population prior to exposure to a virus or other pathogenic agent as an atypical subgroup, 2) classifying similarities or differences in underlying medical treatments that distinguish members of said atypical subgroup to establish outliers or edge cases in the larger exposure group including the presence of excipient EDTA in nebulized medications used to treat respiratory disease, 3) characterize a molecular mechanism or mechanism to explain how said outliers could disrupt or otherwise abrogate the virus infection or other pathogenic process, and 4) utilize knowledge from understanding the molecular mechanism of the outlier to formulate a new therapeutic agent that would include increasing the concentration of Na₂EDTA in nebulizer solutions from about 1.2 to about 2.4 or to about 2.8 mg/mL.

According to another aspect consistent with the principles of the invention, sanitizing compositions are provided which may be used to sanitize objects and surfaces of SARS-CoV-2 coronavirus and other pathogens are provided. In some embodiments, a sanitizing composition may comprise approximately at least 0.07% Na₂EDTA dihydrate by weight, preferably in solution in an alcohol-based solvent, such as ethyl alcohol or isopropyl alcohol. In further embodiments, a sanitizing composition contains at least about 0.07% Na₂EDTA·2H₂O w/w or other edetate in pharmaceutically acceptable and/or hydrate form containing the equivalent molar amount of EDTA. In some of those embodiments the sanitizing composition is a soap or alcohol-base hand sanitizer, lotion, or spray to reduce COVID-19 virus infectivity. The Cosmetic Ingredient Review Expert Panel found that EDTA ingredients are safe as used in cosmetic formulations. The typical concentration of a disodium edetate in cosmetics is less than 2%, and the lowest dose reported to cause a toxic effect in animals was 750 mg/kg/day. (Lanigan R S and Yamarik T A, “Final report on the safety assessment of EDTA, calcium disodium EDTA, diammonium EDTA, dipotassium EDTA, disodium EDTA, TEA-EDTA, tetrasodium EDTA, tripotassium EDTA, trisodium EDTA, HEDTA, and trisodium HEDT”, Int. J. Toxicol. 2002; 21 Suppl. 2:95-142).

In preferred embodiments, a sanitizing composition used for virus disinfectant protection contains at least about 0.07% disodium edetate by weight in an alcohol-based sanitizing composition that is intended for use in conjunction with, or in place of, a traditionally used sanitizing composition, such as soap and water, hand sanitizers, germicidal towelettes, cleaning solutions or sprays. In other preferred embodiments Na₂EDTA·2H₂O is added to a traditional hand sanitizer, germicidal towelette, cleaning solutions or spray so as to contain a final concentration of approximately 1.5 mM EDTA.

In further preferred embodiments, a sanitizing composition may be used in a method of preventing a viral condition in a subject, the method comprising: adding Na₂EDTA·2H₂O, to an alcohol-based hand sanitizer, lotion, spray or soap in effective EDTA molar amount to further reduce or prevent virus infectivity.

Pharmaceutical grade calcium disodium EDTA is FDA approved for the treatment of heavy metal poisoning (particularly lead) and, numerous physicians have utilized pharmaceutical grade disodium edetate with great benefit in several diseases and conditions other than their officially approved use. There are two associations of doctors whose physician members are trained in the administration of disodium edetate for the treatment and prevention of atherosclerosis and other chronic degenerative diseases.

Pharmaceutical grade sodium edetate has been used in chelation therapy for treating ischemic heart disease for more than 50 years (Lamas G A et al., “Effect of disodium EDTA chelation regimen on cardiovascular events in patients with previous myocardial infarction: the TACT randomized trial” JAMA 2013; 309(12), 1241-1250) and the older literature shows that the FDA also approved intravenous EDTA treatment as possibly effective in occlusive vascular disorders, arrhythmias, and atrioventricular induction defects. Additionally Na₂EDTA dihydrate has been added to nebulized bronchodilator solutions in the United States as both non-sterile and sterile-filled products. (Asmus M J et al., “Bronchoconstrictor additives in bronchodilator solutions”, J. Allergy Clin. Immunol. 1999; 104(Pt 2): 553-60). Thus, while no one has previously used pharmaceutical Na₂EDTA dihydrate or other calcium chelating agent to prevent clinical virus infection, the use of pharmaceutical Na₂EDTA to disrupt virus infection as disclosed herein provides new treatment options against coronavirus (CoV) infections and other single stranded RNA viruses having calcium-dependent fusion processes. Thus, the prior art does not identify the specific formulations of Na₂EDTA or combination therapies with a beta-2 agonist that is expected to provide improved therapeutic outcomes for persons infected by, or exposed to, those viruses having calcium-dependent fusion processes.

According to one embodiment consistent with the principles of the invention, pharmaceutically acceptable formulations for the treatment of a viral condition resulting from infection by a single stranded RNA virus, including those caused by a Coronavirus, such as SARS-CoV-2 virus, also known as COVID-19, SARS-CoV-1 virus, HCoV-229E virus, and Rubella virus are provided.

In some embodiments, a pharmaceutically acceptable formulation for the treatment of SARS-CoV-2 viral infection contains at least one calcium chelating agent, or a pharmaceutically acceptable salt and/or hydrate form thereof, in therapeutic effective amount for disrupting calcium-dependent virus cleavage by host protease TMPRSS2 or virus fusion to host cells, wherein the virus is a single stranded RNA virus. The pharmaceutically acceptable formulation is administered by any suitable delivery method, including a route or routes that administer the at least one calcium chelator or a pharmaceutically acceptable salt and/or hydrate form thereof in effective amount for disrupting calcium-dependent virus fusion to host cells. In preferred embodiments, the calcium-dependent virus cleavage by host protease TMPRSS2 inhibitor or virus fusion pathway inhibitor is pharmaceutical grade disodium ethylenediamine tetraacetate dihydrate (Na₂EDTA·2H₂O). In further preferred embodiments, a pharmaceutical composition contains or is configured to provide a dose of between about 1.0 to about 10.0 milligrams of disodium edetate, and more preferably in a dose of between about 2.0 to 3.0 milligrams of disodium edetate or other edetate containing a molar equivalent amount of EDTA or other calcium-dependent inhibitor of host protease TMPRSS2 or other calcium-dependent virus fusion pathway inhibitor in effective amount for said protease or pathway inhibition. In further preferred embodiments, a pharmaceutically acceptable formulation contains or is configured to provide a dose of between about 1.0 to about 4.0 milligrams, and more preferably in a dose of between about 2.0 to about 3.0 milligrams, or about 2.4 milligrams of disodium edetate or other calcium-dependent inhibitor of host protease TMPRSS2 or calcium-dependent virus fusion pathway inhibitor in effective amount for said protease or pathway inhibition, and a beta-2 agonist, preferably a short acting beta-2 agonist, such as albuterol or metaproterenol, preferably in pharmaceutically acceptable salt form.

According to other embodiments consistent with the principles of the invention, methods of treating a viral infection by a single stranded RNA virus is provided. In preferred embodiments, the method of treatment is used to treat a viral condition in a patient, in which the viral condition is caused by a coronavirus, such as SARS-CoV-2 virus, also known as COVID-19, SARS-CoV-1 virus, HCoV-229E virus or other single stranded RNA virus, such Rubella virus. In further preferred embodiments, the method for treating the viral condition in a subject is comprised of administering a therapeutically effective amount of a pharmaceutically acceptable formulation containing a pharmaceutical calcium chelating agent, or pharmaceutically acceptable salt and/or hydrate thereof, such as disodium edetate or hydrate form thereof, whereby the viral condition is ameliorated. In preferred embodiments, a pharmaceutically acceptable formulation for treating single stranded virus infections, including SARS-CoV-2 coronavirus infection containing at least one calcium chelating agent, or a pharmaceutically acceptable salt and/or hydrate form thereof, such as such as Na₂EDTA·2H₂O, is provided. Other preferred embodiments are delivery methods that include a route or routes to administer at least one calcium chelating agent or a pharmaceutically acceptable salt thereof capable of disrupting calcium-dependent virus cleavage by host protease TMPRSS2 or calcium-dependent virus fusion to host cells in a pharmaceutically acceptable formulation. Preferably, the pharmaceutically acceptable formulation further contains one or more pharmaceutically acceptable excipients. More preferably, at least one of those excipients is a carrier.

In some embodiments, a method for the treatment, amelioration or prevention of a condition or disease caused by a single stranded RNA virus infection, such as that caused by SARS-CoV-2 coronavirus comprises administering to a subject in need thereof a therapeutically effective amount of a combination of (i) disrupter of calcium-dependent virus cleavage by host protease TMPRSS2 or calcium-dependent virus fusion to cells of the subject and (ii) a beta-2 receptor agonist known to those skilled in the art for treating human airway disease or for treating symptoms of bronchoconstriction attributable to said disrupter administering.

In some embodiments, a treatment method includes a delivery method that uses an intravenous route to administer at least one calcium chelating agent, or a pharmaceutically acceptable salt and/or hydrate form thereof, capable of disrupting calcium-dependent virus cleavage by host protease TMPRSS2 or calcium-dependent virus fusion to host cells.

In some embodiments, a treatment method includes a delivery method that uses aerosol routes to administer at least one calcium chelator or a pharmaceutically acceptable salt thereof capable of disrupting calcium-dependent virus cleavage by host protease TMPRSS2 or virus fusion to host cells.

In some embodiments, a treatment method includes a delivery method that uses oral (e.g., by mouth) route to administer at least one calcium chelating agent or a pharmaceutically acceptable salt thereof capable of disrupting calcium-dependent virus cleavage by host protease TMPRSS2 or virus fusion to host cells.

In some embodiments, a treatment method includes a delivery method that uses combining inhalation and intravenous routes to administer at least one calcium chelator or a pharmaceutically acceptable salt thereof that can disrupt calcium-dependent virus cleavage by host protease TMPRSS2 or virus fusion.

In some embodiments, a treatment method includes a delivery method that uses combining aerosol, intravenous, and/or oral routes to administer at least one calcium chelator or a pharmaceutically acceptable salt thereof that can disrupt calcium-dependent virus cleavage by host protease TMPRSS2 or virus fusion.

In some embodiments, a treatment method includes a delivery method that uses inhalation of aerosolized drugs as a route to administer at least one calcium chelating agent, or a pharmaceutically acceptable salt and/or hydrate form thereof, capable of disrupting calcium-dependent virus cleavage by host protease TMPRSS2 or virus fusion to host cells.

The following examples demonstrate the functional interaction of calcium chelating agents to prevent single stranded RNA virus infection or otherwise disrupt virus fusion of the single stranded RNA virus with host cells. It will be understood, however, that the exact dose level for any specific patient will depend upon a multiplicity of factors including the affinity of the specific calcium chelating agent for Ca²⁺, the age, body weight, general health, sex, diet, time and route of administration, rate of elimination, drug combinations and the severity of the viral infection.

Inhalation of nebulized solutions are capable of delivering aerosolized Na₂EDTA directly into the very tissues attacked by COVID-19: lung alveoli, bronchi, larynx mouth nose, pharynx and throat. Aerosolized Na₂EDTA in hydrate form is delivered by any of the three main inhalation systems: pressurized metered-dose inhalers (MDIs), dry powder inhalers (DPIs), and nebulizers. Prior human inhalation studies with EDTA solutions (Hoffmann M, Cell 2020; Wan Y, J. Virol 2020, op.cit.) using a nebulizer to aerosolize dissolved Na₂EDTA·2H₂O provides guidance on how to directly deliver Na₂EDTA in hydrate form for treating or attenuating SARS-CoV-2/COVID-19 infection in affected tissues. However, inhalation therapy of a nebulized Na₂EDTA hydrate solution also requires the patient be monitored for potential bronchoconstriction and a beta-2 agonist, including albuterol drug treatment concurrently available to alleviate any potential bronchospasm.

In preferred embodiments, a treatment method includes delivery of an aerosolized edetate solution to the lower respiratory tracts of SARS-CoV-2/COVID COVID-19 patients on mechanical ventilators. To achieve this goal, either jet or ultrasonic nebulizers are preferably used for delivering aerosols to mechanically ventilated patients. Nebulizers can be attached in the inspiratory limb of the ventilator circuit or at the patient Y-piece. Placing the jet nebulizer at a distance from the endotracheal tube has better efficiency than placing it between the patient Y-piece and the endotracheal tube, because the ventilator circuit acts as a spacer for aerosol to collect between inspirations. The patients on mechanical ventilators are very vulnerable and require especially diligent monitoring of the patient for potential bronchoconstriction. Accordingly, a beta-2 agonist, such as albuterol, or other bronchodilator, should be available concurrently to alleviate any potential bronchospasm.

In other preferred embodiments, a treatment method includes an inhalation delivery method for patients not on ventilators such as delivery by a standalone nebulizer to aerosol an edetate solution, preferably a disodium edetate solution at the concentration of from about 1.2 to about 12.8 mg/mL or other edetate salt of hydrate thereof containing an equimolar amount of EDTA. Like patients on mechanical ventilators, administer of an aerosolized Na₂EDTA·2H₂O solution requires diligent monitoring of the patient for potential bronchoconstriction. Accordingly, a beta-2 agonist, such as albuterol, should available to alleviate any potential bronchospasm. Thus a supplementary treatment option is provided when in-patient beds become overloaded with SARS-CoV2/COVID-19 patients.

In preferred embodiments, a treatment method includes the delivery of intravenous disodium edetate not unlike the randomized controlled trials of calcium chelation in patients with chronic lead poisoning, coronary artery disease and chronic renal insufficiency. Accordingly, one example of the current invention is the administration of an intravenous sodium edetate solution to a human subject in need thereof, preferably by a dose of 40 mg/kg body weight over a 3-hour period with administration of that dose at least three times per week for a duration of 33 days or more.

In some embodiments, a treatment method includes therapy for treating a gastrointestinal viral infection caused by SARS-CoV-2/COVID-19. Namely, in some of those embodiment patients having the gastrointestinal viral infection are treated by consumption of food-additive grade EDTA containing a dose of 800 mg of disodium edetate daily. An oral dose of disodium edetate that is available as a stabilizer in food supplements typically results in about 40 mg EDTA being absorbed each day based on a 1954 study showing that the human body absorbs a maximum of 5% of orally administered EDTA. (Foreman H and Trujillo T, “The metabolism of C14 labeled ethylenediaminetetraacetic acid in human beings”, J. Lab. Clin. Med. 1954 April; 43(4):566-71).

The pharmaceutically acceptable formulations of the invention, in various aspects, are useful in treatment methods described herein and are administered by injection, or prepared for oral, pulmonary, nasal or for any other form of administration. Preferably, to enable local contact with infected host tissues in the respiratory tract, the pharmaceutically acceptable formulations are administered, for example, by aerosol administration through inhalation. The mode of administration for the most effective response needs to be determined empirically and the means of administration described below are given as examples, and do not limit the method of delivery of the pharmaceutically acceptable formulations of the present invention in any way.

The particular dosage form of a pharmaceutically acceptable formulation provided by the present invention in some embodiments further comprise a vial, ampule, container, capsule or tablet containing the pharmaceutically acceptable formulation together with dosage instructions for the administration of the dosage form to the patient for the treatment, attenuation, or prevention of a viral disease as described herein.

Where logistically possible, and as tolerated by the patient, the present invention contemplates pulmonary delivery of the compounds. Here, the compounds may be delivered by inhalation to the lungs and respiratory track of a patient in need as it traverses through the respiratory tree to lung epithelial lining.

Devices for pulmonary delivery contemplated for use in the practice of this invention are a wide range of mechanical devices that are designed for the pulmonary delivery of therapeutic products. Those include, but are not limited to metered-dose inhalers, nebulizers, powder inhalers and nebulizer attachments to mechanical ventilators, which preferably are those familiar to one skilled in the art.

According to another aspect consistent with the principles of the invention, a drug discovery method is provided which utilizes the characterization of a molecular mechanism to explain unforeseen clinical co-morbidly patterns identified as outliers derived from understanding the molecular mechanism of the outlier to formulate a new therapeutic regimen. In preferred embodiments, the drug discovery method utilizes the characterization of a molecular mechanism of an unforeseen clinical co-morbidly patterns identified as outliers utilize knowledge from understanding the molecular mechanism of the outlier to formulate the new therapeutic regimen, including a therapeutic regimen that uses a calcium chelating agent for the treatment of a single stranded RNA infection.

In some embodiments, the drug discovery method includes the steps of: 1) identifying outliers of unexpected disease pattern(s) in the co-morbidity data reflecting the expected disease prevalence for a matched population prior to exposure to a virus or other pathogenic agent as an atypical subgroup, 2) classifying similarities or differences in underlying medical treatments that distinguish members of said atypical subgroup to establish outliers or edge cases in the larger exposure group including the presence of excipient EDTA in nebulized medications used to treat respiratory disease, 3) characterize a molecular mechanism or mechanism to explain how said outliers could disrupt or otherwise abrogate the virus infection or other pathogenic process, and 4) utilize knowledge from understanding the molecular mechanism of the outlier to formulate a new therapeutic agent that would include a concentration of disodium edetate in nebulizer solutions that has been increased from excipient levels to a therapeutically effective level in the range of between about 0.5 to about 10.0 milligrams in about a 2.5 mL solution, preferably about 2.4 mg in solution volume of about 2.5 mL or other edetate solution in equimolar EDT A concentration.

### NUMBERED EMBODIMENTS

The following numbered embodiments are for illustrative purposes and are not intended to constrain the overall scope of the invention.
1. A pharmaceutically acceptable formulation comprised or consisting essentially of: a) calcium chelating agent, or pharmaceutically acceptable salt and/or hydrate form thereof, as an active pharmaceutical ingredient of the formulation; b) one or more pharmaceutically acceptable excipients; and c) optionally a beta-2 agonist, or a pharmaceutically acceptable salt and/or hydrate form, as another active pharmaceutical ingredient of the formulation.
2. The pharmaceutically acceptable formulation of embodiment 1, wherein the calcium chelating agent, or pharmaceutically acceptable salt thereof, is a calcium-dependent virus fusion pathway inhibitor.
3. The pharmaceutically acceptable formulation of embodiment 1 or 2, wherein the calcium chelating agent, or pharmaceutically acceptable salt and/or hydrate form thereof, is an inhibitor of a protease responsible for cleavage of a Coronavirus spike protein.
4. The pharmaceutically acceptable formulation of embodiment 3, wherein the protease is TMPRSS2.
5. The pharmaceutically acceptable formulation of any one of embodiments 1-4, wherein the calcium chelating agent is pharmaceutical grade EDTA, and wherein its pharmaceutically acceptable salt and hydrate form is preferably Na₂EDTA·2H₂O.
6. The pharmaceutically acceptable formulation of any one of embodiments 1-5, wherein one or two of the pharmaceutically acceptable excipients are carriers, wherein the carrier(s) provides a dry powder inhalant.
7. The pharmaceutically acceptable formulation of embodiment 6, wherein the carrier providing the dry powder inhalant is lactose monohydrate.
8. The pharmaceutically acceptable formulation of embodiment 6, wherein the carriers providing the dry powder inhalant are lactose monohydrate and magnesium stearate.
9. The pharmaceutically acceptable formulation of any one of embodiments 1-5, wherein one or two of the pharmaceutically acceptable excipients are diluents, wherein the diluent(s) provides a solution or suspension capable of nebulization.
10. The pharmaceutically acceptable formulation of embodiment 9, wherein the diluent providing the solution or suspension capable of nebulization is water for injection.
11. The pharmaceutically acceptable formulation of embodiment 9, wherein the diluents providing the solution capable of nebulization are water for injection and ethanol or water for injection and propylene glycol.
12. The pharmaceutically acceptable formulation of any one of embodiments 1-11, wherein the beta-2 agonist is present in the formulation.
13. The pharmaceutically acceptable formulation of embodiment 12, wherein the beta-2 agonist is a short acting beta-2 agonist.
14. The pharmaceutically acceptable formulation of embodiment 13, wherein the short acting beta-2 agonist is albuterol or metaproterenol and wherein its pharmaceutically acceptable salt form is albuterol sulfate or metaproterenol sulfate, respectively.
15. The pharmaceutically acceptable formulation of any one of claims 1-14, wherein the formulation is a solution in unit dosage form having between about 1.0 to about 10.0 milligrams pharmaceutical grade Na₂EDTA dihydrate dissolved in about 2.5 mL diluent or having an equimolar concentration of EDTA.
16. The pharmaceutically acceptable formulation of embodiment 15, wherein the solution in unit dosage has between about 2.0 to about 3.0 milligrams Na₂EDTA dihydrate dissolved in about 2.5 mL diluent or having an equimolar concentration of EDTA, and a short acting beta-2 agonist.
17. The pharmaceutically acceptable formulation of embodiment 16, wherein the short acting beta-2 agonist is in pharmaceutically acceptable salt form wherein the pharmaceutically acceptable salt is albuterol sulfate.
18. The pharmaceutically acceptable formulation of any one of embodiments 1-5, wherein the pharmaceutically acceptable excipients provide an alcohol-based sanitizer, lotion or spray or a soap.
19. The pharmaceutically acceptable formulation of any one of embodiments 1-5, wherein the pharmaceutically acceptable excipients provides an alcohol-based sanitizer containing 0.07% disodium edetate by weight.
20. The pharmaceutically acceptable formulation of embodiment 18 or 19, wherein the alcohol of the alcohol-based sanitizer is ethyl alcohol or isopropanol.
21. A method of treating or preventing a viral infection in a subject, wherein the viral infection is through fusion of a virus to cells of the subject by a calcium-dependent pathway, the method comprising the step of: administering to the subject a therapeutically effective amount of a pharmaceutically acceptable formulation of any one of embodiments 1-20.
22. The method of embodiment 21, wherein said administering is by inhalation of the pharmaceutically acceptable formulation of any one of embodiments 6-17.
23. The method of embodiment 22, wherein the beta 2-agonist is present in the formulation.
24. The method of embodiment 21, 22 or 23, wherein the virus is a single stranded RNA virus.
25. The method of embodiment 24, wherein the single stranded RNA virus is Rubella virus, a α-Coronavirus or a β-Coronavirus.
26. The method of embodiment 24, wherein the single stranded RNA virus is a α-Coronavirus.
27. The method of embodiment 26, wherein the α-Coronavirus is HCoV-229E.
28. The method of embodiment 24, wherein the single stranded RNA virus is a β-Coronavirus.
29. The method of embodiment 28, wherein the β-Coronavirus is SARS-CoV-2 virus, also known as COVID-19, or SARS-CoV-1 virus.
30. The method of any one of embodiments 21-29, wherein said administering is by inhalation of the pharmaceutically acceptable formulation of claim 9, wherein the beta-2 agonist is not present and wherein the method further comprises co-administering to the subject a short acting beta-2 agonist in effective amount for reducing bronchoconstriction from said inhalation administering.
31. A vaccine composition, the vaccine composition comprising a live, attenuated coronavirus comprising a SARS-CoV-2 variant in which the SARS-CoV-2 fusion loops (amino acids 816-855) are replaced with the less pathogenic HCoV-229E fusion loop (amino acids 923-982) to provide substantially the same antigenicity of SARS-CoV-2 while limiting its pathogenicity to no more than the level of HCoV-229E.

Although the present invention has been illustrated and described herein with reference to preferred embodiments and specific numbered thereof, it will be readily apparent to those of ordinary skill in the art that other embodiments and examples are capable for achieving similar functions and/or like results. All such equivalent embodiments and examples are within the spirit and scope of the present invention, are contemplated thereby, and are intended to be covered by the claims.

### EXPERIMENTAL

### Calcium-Dependent Fusion Process Required for Viral Infection

Even now, the actual mechanism of virus membrane fusion is not completely understood. In the case of Coronaviruses (CoVs), it is not a simple two-step process of receptor binding (via the S1 domain) and membrane fusion (via the S2 domain containing the fusion peptide). Viral entry into host cells requires that there is a domain of the S protein that interacts with opposing hydrophobic cellular membranes called a fusion peptide or fusion loop. These fusion peptides (fusion loops, FL) are generally external amino acid domains that insert into the host membranes after major conformational changes of the virus S protein following proteolytic cleavage to initiate the process fusion with the host membrane.

When the S1/S2 site and S2′ are activated by host proteases (e.g., TMPRSS2) there are changes in the cleavage site position relative to the fusion peptide to modulate the fusion loop (FL). This process gives CoVs the unique flexibility to invade different cell types and host species. Additionally, the CoVs fusion process employs a calcium-dependent fusion process that was only recently discovered for Rubella and later described for SARS-CoV-1 infection. (Lai A L et al., “The SARS-CoV fusion peptide forms an extended bipartite fusion platform that perturbs membrane order in a calcium-dependent manner”, J. Mol. Biol. 2017 Dec. 8; 429(24):3875-3892).

While two fusion peptides (FLs) were found with SARS-CoV-1, influenza had no calcium-dependent membrane fusion process. The calcium dependent membrane-ordering results in more effective binding that can penetrate deeper into membranes. There are two FL domains in each SARS-CoV versus a single FL domain found in for HCoV-229E and Rubella shown in Table 2 below. Thus, this calcium-dependent requirement for the FL process may explain both the increased lethality of the beta CoVs and the apparent resistance of asthma patients to SARS-CoV-2 infection due to inhaling medications containing EDTA excipients.

Computer analysis comparing SARS-Cov-1 to of SARS-Cov-2 using protein Blast revealed two calcium binding domains in SARS-Cov-19 that a similar to the new calcium dependent fusion mechanism recently discovered for Rubella. Further computer analysis revealed that the less pathogenic alpha Coronal Virus H229E has a calcium binding site which is not unlike the homology the Rubella sequence. Analysis of Human protease literature show a Calcium binding domain required for COVID-19 (SARS-CoV-2) infection. The substrate binding of proteins with calcium binding sites similar to the host protease TMPRSS2 were disrupted by EDTA as published in 2020 (Lanigan R S, Int. J. Virol. 2002, op. cit.). Molecular analysis of both the virus (computer sequence) and the required host activation process by protease, TMPRSS2 show that EDTA can disrupt two key steps in the infection process. That is EDTA is capable of disrupting two different targets. However, to be optimally efficacious against a single stranded RNA virus infection, EDTA is used in methods of the present application at higher concentrations (e.g., 2.4 mg/mL, which is higher than excipient levels in formulation of the beta-2 agonist metaproterenol). Therefore, the observation that the Asthma/COPD patient subgroup appeared to be “resistant” to COVID-19 infection is now recognized by the present invention to be due to the EDTA excipient contained in asthma medications (e.g. Metaproterenol) as a result of the computer analysis described herein showing there is a key calcium requirement for COVID-19 infections.

The WHO has classified the severe acute respiratory syndrome coronavirus 2 (SARS-CoV-2), newly named COVID-19, as a R CoV of group 2B. (Hui D S et al. “The continuing 2019nCoV epidemic threat of novel coronaviruses to global health—The latest 2019 novel coronavirus outbreak in Wuhan, China”, Int. J. Infect. Dis. 2020; 91: 264-266). Sequence results from patient isolates show the new beta-CoV-2 strain have 99.8-99.9% nucleotide identity. (Zhou P et al., “A pneumonia outbreak associated with a new coronavirus of probable bat origin”, Nature 2020; 579(7798):270-273). While the overall genetic sequence of the COVID-19 (SARS-CoV-2) has only 80% identity to SARS-CoV, (Dubé M, et al., PLoS Pathog. 2014, op. cit.) there are two calcium dependent binding domains, that act as fusion loops (FL), in the COVID-19's spike (S) surface protein that are almost 100% homologous to the SARS-CoV fusion domains and appear critical for virus entry into a host. (Lai A L et al., J. Mol. Biol. 2017, op. cit.). According to NCIB protein Blast analysis there is also a significant homology of Rubella Virus membrane glycoprotein E2 amino acids 49 to 55 with the SARS-CoV-2 fusion loop 2 as shown in Tables 1 and 2. The mechanism of the new therapy targets the calcium-dependent infectious process that was recently described in 2014, when Rubella Virus, an airborne pathogen, was the first virus discovered to have a calcium-requiring viral fusion protein. (Yin Y and Wunderink R G, Respirology_2018, op. cit.; Dubé M, et al., PLoS Pathog. 2014, op. cit.; DuBois R M et al. “Functional and evolutionary insight from the crystal structure of rubella virus protein E1”, Nature 2013, Jan. 24; 493(7433):552-6). Importantly, this Rubella research demonstrated that the addition of the calcium chelator, EDTA completely abrogated Rubella fusion, and prevented Rubella virus infection, in the “test tube” (Yin Y and Wunderink R G, Respirology_2018, op. cit.).

Results of the novel protein sequence analysis is shown in Table 1 and Table 2 which exhibit the Protein Blast Alignment Tool data from the calcium binding fusion domains, labeled FL1 and FL2 (Totura A L et al. Expert Opin. Drug Discov. 2019, op cit.), respectively, that compare the spike proteins of COVID-19 (SARS-CoV-2) with SARS-COV-1 and Rubella utilizing cited reference data; GenBank: QHD43416.1 (CoV-2), NCBI Reference Sequence: NP_828851.1(CoV), and GenBank: ACN50046.1. (Rubella) (Boratyn G M et al. “BLAST: a more efficient report with usability improvements”, Nucleic Acids Res. 2013; 41:W29-33 (Web Server issue)

TABLE 1
pBlast Alignment Metrics

| Fusion Loop | | | | | |
|---|---|---|---|---|---|
| Domains | | | | | |
| Aligned | Score | Expect | Method | Identities | Positives |
| FL1-CoV- | 47.4 bits (111) | 7e−13 | Composition- | 21/22 | 22/22 |
| 1/CoV-2 | | | Based stats | (95%) | (100%) |
| FL2- CoV- | 47.4 bits (111) | 9e−13 | Composition- | 19/22 | 20/21 |
| 1/CoV-2 | | | Based stats | (90%) | (95%) |
| FL2-CoV- | 13.5 bits (23) | 0.99 | Composition- | 4/7 | 5/7 |
| 2/Rubella | | | Based stats | (57%) | (71%) |
| Fl2-CoV-2/ | 14.2 bits (25) | 0.48 | Composition- | 4/6 | 5/6 |
| HCoV-229E | | | Based stats. | (67%) | (83%) |
| Fl2-CoV- | No similarity | NA | Composition- | NA | NA |
| 2/Influenza | | | Based stats. | | |

TABLE 2
pBlast Alignment Amino Acid Locations

| | Fusion Loop Amino Acid | Reference Sequence |
|---|---|---|
| Source | Position | SEQ ID No. |
| FL1 CoV-2 | 816 SFIEDLLFNKVTLADAGFIKQY 837 | GenBank: QHD43416.1 |
| | | SEQ ID: 1 |
| pBlast | SFIEDLLFNKVTLADAGF+KQY | SEQ ID: 9+KQY |
| Alignment | | |
| FL1 CoV | 798 SFIEDLLFNKVTLADAGFMKQY 819 | NCBI Reference Seq.: |
| | | NP_828851.1 |
| | | SEQ ID: 2 |
| FL2 CoV-2 | 835 KQYGDCLGDIAARDLICAQKF 855 | GenBank: QHD43416.1 |
| | | SEQ ID: 3 |
| pBlast | KQYG+CLGDI ARDLICAQKF | SEQ ID: 10+ SEQ ID: 11 |
| Alignment | | SEQ ID: 12 |
| FL2 CoV | 817 KQYGECLGDINARDLICAQKF 837 | NCBI Reference Seq.: |
| | | NP_828851.1 |
| | | SEQ ID: 4 |
| FL2 CoV-2 | 841 GDIAARD 848 | GenBank: QHD43416.1 |
| | | SEQ ID: 5 |
| pBlast | GD + RD | |
| Alignment | | |
| Rubella | 49 GDDSGRD 55 | GenBank: ACN50046.1 |
| | | SEQ ID: 6 |
| FL2 CoV-2 | 835 KQYGDCL 841 | GenBank: QHD43416.1 |
| | | SEQ ID: 7 |
| pBlast | K+YG C | |
| Alignment | | |
| F12 HCoV-229E/ | 923 KRYGFC 928 | GenBank: NP_828851.1 |
| CoV-2 | | SEQ ID: 8 |
| F12-CoV2/ HA | No significant | GenBank: AD177360.1 |
| Influenza | similarity found | |

Here, as shown in Tables 1 and 2, the high peptide homology of FL1 and FL2 domains in COVID-19 compared to SARS-CoV and Rubella provides a target for therapy with a calcium chelating agent to prevent/treat COVID-19 and other single stranded RNA viral infections that substantially advances the art of treating these viral infections.

Ethylenediamine tetraacetic acid (EDTA) was first synthesized in 1935 and has been employed as an excipient in bronchial dilator solutions for decades (e.g., albuterol, netaproterenol). EDTA has been added to nebulized bronchodilator solutions in the United States as both nonsterile and sterile-filled products. (Asmus M J et al., “Bronchoconstrictor additives in bronchodilator solutions”, J. Allergy Clin. Immunol. 1999 August; 104(2 Pt 2): S53-60). Accordingly, EDTA is often present as a preservative or stabilizing agent in nebulizer solutions used to treat asthma and chronic obstructive pulmonary disease (Beasley R, “Effect of EDTA on the bronchodilator response to Duovent nebulizer solution”, N.Z. Med. J. 1989 July 12; 102(871):357) but never as an active therapeutic ingredient. Historically, common nebulizer therapies used by asthma and COPD patients have had EDTA concentrations available in nebulizer solutions that vary from 0.1 to 0.5 mg/mL. (Kamin W et al. “Inhalation solutions: which one are allowed to be mixed? Physico-chemical compatibility of drug solutions in nebulizers”, J. Cyst. Fibros. 2006 December; 5(4):205-13). For example, albuterol sulfate (manufactured by Dey Laboratories) contained 300 μg of EDTA, which is also far below the threshold dose for bronchoconstriction. Currently, Metaproterenol Inhalation Solution USP is formulated with EDTA as a unit-dose bronchodilator to be administered by oral inhalation with the aid of an intermittent positive pressure breathing apparatus (IPPB). It contains 0.4% or 0.6% w/v metaproterenol sulfate in a sterile, acidic, aqueous solution containing edetate disodium dihydrate, sodium chloride, hydrochloric acid, and/or sodium hydroxide for pH adjustment. (see <https://www.drugs.com/pro/metaproterenol.html>

In one study, volunteer subjects received an inhalation challenge with increasing concentrations of EDTA (0.25 to 10.0 mg/mL) in a double-blind fashion. [BMJ-1987](Beasley, C R et al., “Bronchoconstrictor properties of preservatives in ipratropium bromide (Atrovent) nebuliser solution”, Br. Med. J. (Clin Res. Ed.) 1987 May 9; 294(6581): 1197-1198). Here, EDTA produced concentration-dependent bronchoconstriction that did not resolve spontaneously within 1 hour. Mean EDTA PC₂₀ FEV₁ was 2.4 mg/mL (range 1.2 to 12.8 mg/mL). That study concluded there was no significant difference in airway response to EDTA among volunteers receiving beta-2 agonist treatments. To study off-target bronchoconstriction by EDTA, it was found that albuterol sulfate (1 microgram/kg IV) significantly attenuated EDTA-induced bronchoconstriction in canines. (Lindeman K S, et al., “Functional antagonism of airway constriction in the canine lung periphery”, J. Appl. Physiol. 1985.1991 November; 71(5): 1848-55) Additionally, intravenous EDTA chelation therapy has been safely used for more than 50 years. (Lamas G A et al., “Effect of disodium EDTA chelation regimen on cardiovascular events in patients with previous myocardial infarction: the TACT randomized trial”, JAMA 2013 309(12), 1241-1250). There were an estimated 500,000 visits for chelation therapy in the U.S. for 1993 (Grier M T and Meyers D G, “So much writing, so little science: a review of 37 years of literature on edetate sodium chelation therapy”, Ann. Pharmacother. 1993; 27:1504-1509) and 800,000 in 1997. (see NIH RFA <https://grants.nih.gov/grants/guide/rfa-files/RFA-AT-01-004.html> EDTA CHELATION THERAPY FOR CORONARY ARTERY DISEASE, Release Date: Apr. 30, 2001 RFA: RFA-AT-01-004 National Center for Complementary and Alternative Medicine <http://nccam.nih.gov>). A Canadian survey found that 8% of patients who had undergone cardiac catheterization had used chelation therapy. (Knudtson M L et al., “Chelation therapy for ischemic heart disease: a randomized controlled trial”, JAMA. 2002 Jan. 23-30; 287(4):481-6). Recent medical articles indicate that there is markedly lower reported prevalence of asthma and COPD in patients diagnosed with COVID-19. (Halpin D M G et al. “Do chronic respiratory diseases or their treatment affect the risk of SARS-CoV-2 infection?” Lancet Respir. Med. 2020 Apr. 3; pii: S2213-2600(20): 30167-3; Bhatraju P K et al, “Covid-19 in critically ill patients in the Seattle region—Case Series New Engl. J. Med. 2020 Mar. 30).

Asthma is considered a high medical risk factor for susceptibility to SARS-CoV-2/COVID-19 infection, yet asthma is not on the list of top 10 chronic health problems suffered by people who died from SARS-CoV-2/COVID-19. Resolving this paradox requires looking beyond the binary model of a viral receptor-binding domain (RBD) attaching to the ACE-2 receptor. A pBlast analysis revealed that SARS-CoV-2 surface spike protein contains two calcium-dependent fusion domains that were recently discovered SARS-CoV-1. These viral calcium-dependent binding domains can facilitate membrane fusion only after cleavage by the host surface protease TMPRSS2. Importantly, TMPRSS2 also requires calcium for its SRCR (scavenger receptor cysteine-rich) domain and its LDLRA (LDL receptor class A) domain. Thus, the presence of EDTA excipients in nebulized β₂-agonist medicines can disrupt SARS-CoV-2/COVID-19 infection and can explain the asthma paradox. This model validates repurposing EDTA in nebulizer solutions from a passive excipient to an active drug for treating COVID-19 infections. Repurposed EDTA delivery to respiratory tissues at an initial target dose of 2.4 mg EDTA, or an equimolar amount of disodium edetate dihydrate, per aerosol treatment is readily achievable with standard nebulizer and mechanical ventilator equipment. EDTA is therefore a suitable active pharmaceutical ingredient for treating SARS-CoV-2/COVID-19 in consideration of the newly discovered calcium requirements for virus infection and the regular presence of much lower concentrations of EDTA excipients in common asthma medications such as metaproterenol sulfate.

To explain the unexpected observation that asthma patients and chronic obstructive pulmonary disease patients appear resistant to COVID-19, it is postulated that: 1) SARS-CoV-2/COVID-19 has two calcium-dependent fusion peptide/fusion loop (FL) domains of that is highly homologous to SARS-CoV-1; 2) the substrate recognition site(s) for cleavage by the requisite cell surface protease TMPRSS2 have a conserved SRCR (scavenger receptor cysteine-rich) domain and a LDLRA (LDL receptor class A) domain that utilize calcium to mediate binding to the SARS-CoV-2 (COVID-19) spike protein (i.e., the ligand); and 3) SARS-CoV-2 (COVID-19) infection is, and has been disrupted by exposure to calcium chelating agents such as EDTA in nebulizer medications inhaled by asthma patients to either directly interrupt the cleavage of the S protein by TMPRSS2 and/or impede the calcium-dependent fusion of SARS-CoV-2 (COVID-19) virions with the host membrane via FL1 and FL2 peptide domains.

Searching for the viral mechanisms to elucidate why asthma patients appear resistant to COVID-19 infection uncovered evidence for the key role of calcium in SARS-CoV-2/COVID-19 infection. First, new computer sequence analysis of SARS-COV-2/COVID-19 revealed two unrecognized calcium-dependent fusion loop domains. Second, the substrate recognition sites for the requisite cell surface protease TMPRSS2 have a conserved SRCR (scavenger receptor cysteine-rich) domain and a LDLRA (LDL receptor class A) that utilize calcium. Analysis of nebulizer solutions typically used by asthma patients, for example Metaproterenol, revealed the presence of an excipient called EDTA, a calcium-chelating agent. Triangulating these, the data converges on the previously unrecognized critical importance calcium for effective SARS-CoV-2/COVID-19 infection, and how calcium chelation by EDTA may prevent infection. Accordingly, repurposing EDTA from excipient to therapeutic nebulized drug with or without beta-2 agonist supplementation becomes a new treatment for COVID-19/SARS-CoV-2 patients.

The potential to utilize EDTA to both reduce COVID-19 transmission and treat infection through relatively safe modalities that include nebulizer or mechanical ventilator misting of EDTA solutions (in conjunction with Albuterol/Metaproterenol to minimize bronchoconstriction if needed) and adding EDTA to hygienic products is described herein. As either an “Off-Label” or formal IRB protocol, the clinician measures the clinical response of nebulized pharmaceutically sterile Na₂EDTA·2H₂O dissolved in normal saline at a range up to about 1.2 to about 12.8 mg/mL for the treatment of COVID-19 patients on respirators or with stand-alone nebulizers. When administering nebulized EDTA, the clinician should monitor for signs of bronchial constriction, and administer albuterol or other short acting beta-2 agonist as needed. Patients not on a respirator are similarly treated with an EDTA solution through a nebulizer facemask preferably under direct medical supervision, and if tolerated, the more stable patients are treated at home or as outpatient with a nebulizer mask and an EDTA solution.

The effect of a pharmaceutical composition, having at least one calcium chelator or a pharmaceutically acceptable salt thereof capable of disrupting calcium-dependent virus cleavage by host protease TMPRSS2 or virus fusion to host cells, are tested in humans who are Covid-19/SARS-CoV-2 rtPCR positive by performing a prospective, randomized, placebo-controlled study to compare the effect of administering nebulizer treatments with either containing saline solutions of disodium edetate exclusively or disodium edetate/beta-2 agonist (e.g., albuterol sulfate or metaproterenol sulfate. The focus of the study is directed towards utilizing objective parameters that distinguish treatment groups. Parameters include, for example rtPCR results, clinical progress, and/or length of hospital stay. Moreover, outpatient studies are possible. That is, after initial medical supervision, the less clinically impaired Covid-19/SARS-CoV-2 rtPCR positive patients are treated as an outpatient if the patient tolerates Na2EDTA inhalation therapy. Clinical measurements that are followed in clinic settings include vital signs, FEV1, and pulse oximetry measurements.

Concentrated Na₂EDTA solutions suitable for use in nebulizers and mechanical ventilators can be used to directly test that EDTA can inhibit SARS-CoV-2 infection with relative safety based on prior studies of asthmatics and the long history of adding EDTA to nebulizer treatments (see PubChem Ethylenediaminetetraacetic acid disodium salt (Compound) at <https://pubchem. ncbi.nlm.nih.gov/compound/Ethylenediamine tetraacetic-acid-disodium-salt #section=Absorption-Distribution-and-Excretion>). While the lung airways of asthmatics are sometimes more sensitive to the bronchoconstriction effects of EDTA, prior studies on asthmatics indicate that 2.4 mg/mL of sodium edetate causes, on average a tolerable 20% drop in FEV1 and adding concentrated Na₂EDTA·2H₂O to standard Albuterol nebulizer set-up is expected to mitigate most EDTA induced bronchoconstriction. As a treatment example, assuming the volume from a standard dropper is approximately 0.06 mL and a 0.5 M disodium edetate concentrate, 2 drops of the concentrated 0.5M solution, which contains about 23 to about 26 mg of disodium edetate dihydrate, is added to 2.5 mL of nebulizer diluent (saline or saline/albuterol) and results in about 2.8 mg of EDTA per treatment. Nebulizer treatments are repeatable in accordance with the chosen beta-2 agonist protocol or as tolerated. The 0.5M EDTA concentrate is prepared as follows; Add 186.1 μg of disodium ethylene tetraacetate dihydrate to 800 mL of H₂O. Stir vigorously on a magnetic stirrer. Adjust pH to 8.0 with NaOH (~20 μg of NaOH pellets). Dispense into aliquots and sterilize by autoclaving. It is noteworthy that the disodium salt of EDTA will not go into solution until the pH of the solution is adjusted to ~8.0 by the addition of NaOH. Additionally, an “Off-Label” use of an FDA approved parenteral EDTA drug for nebulizer includes dilution of 200 mg/mL Versenate (edetate calcium disodium injection, USP) to achieve a target dose of 2.4 mg calcium disodium EDTA per aerosol treatment with or without a beta-2 agonist as tolerated. Treatment by inhalation of a calcium chelating agent solution as described inhibits calcium-dependent virus cleavage by host protease TMPRSS2 or the calcium-dependent virus fusion pathway thereby inhibits viral infection of the susceptible host cells.

One illustration demonstrating how to utilize a device for pulmonary administration includes placing the calcium chelating agent Na₂EDTA·2H₂O at a concentration of between about 1.2 to about 12.8 mg/mL, or other calcium chelating agent in equimolar concentration, into a jet nebulizer connected to a bucco-nasal facial mask positioned on the patient and driven with 6 L/min of non-heated and non-humidified pressurized air (Cirrus2 nebulizer and Adult EcoLite™ Aerosol Mask, both from Intersurgical, Wokingham, UK). Upon administration of the EDTA solution, the patient is monitored for potential bronchial spasm. Should bronchial spasm develop, the practitioner administers a β₂ (beta₂) adrenergic receptor agonist, such as albuterol sulfate, to dilate the bronchial passages. albuterol (USAN) is available for nebulizer treatment, along with Salbutamol (INN), formoterol and other β₂ agonists that are also available in solution form for nebulization. The nebulizer form is as effective as administering the drug intravenously. Salbutamol and terbutaline are also available in oral forms and in intravenous forms.

Other examples of commercially available devices suitable for the practice of this invention are, for example and without limitation, the Acorn II nebulizer, manufactured by Marquest Medical Products, Englewood, Colo.; the Ultravent nebulizer, manufactured by Mallinckrodt, Inc., St. Louis, Mo.; the Spinhaler powder inhaler, manufactured by Fisons Corp., Bedford, Mass and the Ventolin metered dose inhaler, manufactured by Glaxo Inc., Research Triangle Park, North Carolina; These devices require the use of pharmaceutical formulations suitable for the dispensing of the compounds. Typically, each formulation is specific to the type of device employed and may include the use of an appropriate propellant material, in addition to the normal diluents, adjuvants and/or carriers useful in therapy. Normally the formulations suitable for use with a nebulizer, either jet or ultrasonic, will characteristically comprise the compounds suspended in water.

It will be understood that in certain aspects, the medicines of the invention used in treatment methods described herein may be given as a single dose schedule, or preferably, in a multiple dose schedule with 1 to 10 separate doses. The dosage schedule will also, at least in part, be governed by the needs of the individual and the clinical judgment of the practitioner. Drug dosage for use with mechanical ventilators or use with standalone nebulizers to aerosol Na₂EDTA·2H₂O preferably under direct medical supervision is at the concentration of 1.2 to 12.8 mg/mL. Additionally, immediate access to a beta-2 agonist should be available to treat potential bronchial spasm.

In addition to inhaled delivery, the medicines of the invention may be administered by any parenteral techniques such as subcutaneous, intravenous and intraperitoneal injections. The medicinal forms suitable for injectable use optionally include sterile aqueous solutions (where water-soluble). Here sterile injectable solutions are formulated by incorporating the active compounds in the required amount in an appropriate solvent with ingredients detailed above, as required, and then sterilized using a microspore filtration. Typically, the dispensing fluid is prepared by incorporating the various sterilized active ingredients into a sterile vehicle that contains the basic dispersion medium and the required other ingredients from those enumerated above.

Intravenous or oral dosage levels of the compounds of the invention will usually be of the order of about 40 mg Na₂EDTA·2H₂O per kilogram body weight, with a preferred dosage range between about 10 mg to about 40 mg per kilogram body weight per day (from about 1.0 g to about 3 g per patient per day).

Also contemplated for use herein are oral solid dosage forms, which are described generally in Martin, Remington's Pharmaceutical Sciences, 18th Ed. (1990 Mack Publishing Co. Easton Pa. 18042) at Chapter 89, which is herein incorporated by reference. Solid dosage forms include capsules, pills, tablets, or other suitable delivery oral vehicles. Such oral dosage forms are preferred for treating gastrointestinal infections by a single stranded RNA virus, which includes SARS-CoV-2/COVID-19

According to another aspect consistent with the principles of the invention, vaccine compositions are provided which may be used to provide humoral immunity against SARS-CoV-2 are provided. In some embodiments, a vaccine composition may comprise a live, attenuated coronavirus comprising a variant in one or more of the amino acids in one or more of the calcium binding fusion loops of the virus spike protein in the region near amino acids 816 through 855 is changed, including replacing SARS-CoV-2 fusion loops (amino acids 816-855) with the less pathogenic HCoV-229E fusion loop (amino acids 923-982) to provide the antigenicity of COVID-19 while limiting the pathogenicity to the level of HCoV-229E The modified coronavirus may be used in a vaccine composition for treating and/or preventing a disease, such as infectious bronchitis, COVID-19, etc., in a subject. Generally, replacement of SARS-CoV-2 S protein amino acids 816 to 855 with HCOV-229E amino acids 923 to 928 provides a live attenuated SARS-CoV-2 hybrid strain suitable for vaccination to generating protective antibodies. An alpha-beta hybrid virus replacing F1 and F2 fusion loops with HCoV-229E amino acids 923-982 is expected to maintain the AEC-2 tissue specificity and host range, yet it will effectively disturb fusion loop mechanism so as to reduce the pathogenicity of SARS-CoV-2 to the level of HCoV-229.

HCoV-229E was discovered in 1966 and is a less pathogenic Alpha coronavirus that appears to have crossed species barriers to infect humans decades or centuries ago. Like SARSCoV-2, HCoV-229E enters the cell via TMPRSS2 to infect humans. However, HCoV-229 is missing the SARS F1 fusion loop, which is comparable to the single FL2 shown in the less pathogenic Rubella virus in Table 2. An attenuated SARS-CoV-2 virus suitable for a vaccine can be created by replacing SARS-CoV-2 amino acids 816-855 with HCoV-229E amino acids 923-982. This replacement will maintain the AEC-2 tissue specificity and host range, yet it will effectively disturb fusion loop mechanism reduce the pathogenicity of SARS-CoV-2 to the level of HCoV-229E. An alpha-beta hybrid virus may be immunologically necessary, since prior studies have shown that immunity after inoculation to HCoV-229E may disappear within a year. Although surveys for 229E antibodies in adults in the United States range from 19 to 41%, there are many individuals, who despite 5 possessing an anti-HoCV-229E antibody, can subsequently experience reinfection and illness. (Monto A S et al. “Coronaviruses viral infections of humans” 2014 Feb. 27: 199-223). In fact, one study found a 66% reinfection rate in volunteers re-exposed to 229E after a year. (Callow, K A et al., “The time course of the immune response to experimental coronavirus infection of man”, Epidemiol. Infect. 1990 105435-446). The significant coronavirus 229E reinfection data both underscores the challenges of finding an effective vaccine, and raises serious questions about the underlying clinical premise(s) that justify the use of “immunity” cards. U.S. Considering COVID-19 Immunity Cards: What Does It Mean?<https://heavy.com/news/2020/04/coronavirus-immunity-cards/>).

The NCBI pBlast tool was used to test the hypothesis that SARS-CoV-2 contains a calcium-dependent fusion domain(s) similar to those that were recently discovered in both Rubella and SARS-CoV-1. An Amino Acids comparison of the two relevant amino acid regions in S protein of SARS-CoV-1 representing fusion loop 1 (FL1=Amino Acids 798-819) and fusion loop 2 (FL2=Amino Acids 835-855) was conducted using the Protein Blast program from the National Center for Biotechnology Information. (Boratyn G M et al., Nucleic Acids Res. 2013, op. cit.) Specifically, Table 1 and Table 2 exhibit the data from the Protein Blast Alignment Tool data from the calcium binding fusion domains, labeled FL1 and FL2, respectively, that compare the spike proteins of COVID-19 (SARS-CoV-2) with SARS-COV and Rubella utilizing cited reference data; GenBank: QHD43416.1 (CoV-2), NCBI Reference Sequence: NP_828851.1(CoV-1), GenBank: ACN50046.1. (Rubella), GenBank: NP_828851(Human coronavirus229E) and GenBank: AD177360.1 (hemagglutinin [Influenza A virus (A/Boston/136/2009(H1N1))]).

The results demonstrated a 100% and a 95% correspondence respectively between the postulated FL1 and FL2 domains in SARS-CoV-2 (COVID-19) compared to the known FL regions for SARS-CoV-1 described in 2017. Additionally, the less pathogenic Alpha coronavirus 229E (HCoV-229E) has a solitary FL2 domain and a reduced homology length compared to the SARS-CoV-2 (COVID-19) FL2. Similarly, amino acids 49 to 55 of the Rubella Virus membrane glycoprotein E2 virus also have a smaller, but significant homology with the FL2 domain. In contrast, the Influenza H1N1 hemagglutinin (HA) protein has no significant similarity to any of the CoV FL domains. The reduced homology of HCoV-229E's single calcium-binding domain to SARS-CoV-2 suggests attenuation of HCoV-229E, which is consistent with HCOV-229E having crossed species barriers to infect humans decades or centuries ago. (Fung S Y et al., “A tug-of war between severe acute respiratory syndrome coronavirus 2 and host antiviral defense: lessons from other pathogenic viruses” Emerg. Microbes Infect. 2020 Mar. 14; 9(1):558-570)

Accordingly, analysis of the different fusion loop homologies suggests replacing SARS-CoV-2 amino acids 816-855 with HCoV-229E amino acids 923-982 may be candidate for an attenuated alpha-beta SARS-CoV-2/COVID-19 vaccine that would be able to generate host immunity, yet lower the pathogenicity to level of HCoV-229E.

What is claimed is:

1. A pharmaceutically acceptable formulation consisting essentially of:
a) calcium chelating agent, or pharmaceutically acceptable salt and/or hydrate form thereof, as an active pharmaceutical ingredient of the formulation;
b) one or more pharmaceutically acceptable excipients; and
c) optionally a beta-2 agonist, or pharmaceutically acceptable salt and/or hydrate form thereof, as another active pharmaceutical ingredient of the formulation.

2. The pharmaceutically acceptable formulation of claim 1, wherein the calcium chelating agent, or pharmaceutically acceptable salt and/or hydrate form thereof, is a calcium-dependent virus fusion pathway inhibitor.

3. The pharmaceutically acceptable formulation of claim 1, wherein the calcium chelating agent, or pharmaceutically acceptable salt and/or hydrate form thereof, is an inhibitor of a protein protease responsible for cleavage of a Coronavirus spike protein.

4. The pharmaceutically acceptable formulation of claim 3, wherein the protease is TMPRSS2.

5. The pharmaceutically acceptable formulation of claim 1, wherein the calcium chelating agent is pharmaceutical grade EDTA and wherein its pharmaceutically acceptable salt and hydrate form is sodium edetate dihydrate (Na₂EDTA·2H₂O).

6. The pharmaceutically acceptable formulation of claim 1, wherein one or two of the pharmaceutically acceptable excipients are carriers, wherein the carrier(s) provides a dry powder inhalant.

7. The pharmaceutically acceptable formulation of claim 6, wherein the carrier providing the dry powder inhalant is lactose monohydrate.

8. The pharmaceutically acceptable formulation of claim 6, wherein the carriers providing the dry powder inhalant are lactose monohydrate and magnesium stearate.

9. The pharmaceutically acceptable formulation of claim 1, wherein one or two of the pharmaceutically acceptable excipients are diluents, wherein the diluent(s) provides a solution or suspension capable of nebulization.

10. The pharmaceutically acceptable formulation of claim 9, wherein the diluent providing the solution or suspension capable of nebulization is water for injection.

11. The pharmaceutically acceptable formulation of claim 9, wherein the diluents providing the solution capable of nebulization are water for injection and ethanol or water for injection and propylene glycol.

12. The pharmaceutically acceptable formulation of any one of claims 1-11, wherein the beta-2 agonist, or pharmaceutically acceptable salt and/or hydrate form, is present in the formulation.

13. The pharmaceutically acceptable formulation of claim 12, wherein the beta-2 agonist is a short acting beta-2 agonist.

14. The pharmaceutically acceptable formulation of claim 13, wherein the short acting beta-2 agonist is albuterol or metaproterenol and wherein its pharmaceutically acceptable salt form is albuterol sulfate or metaproterenol sulfate, respectively.

15. The pharmaceutically acceptable formulation of claim 1, wherein the formulation is a solution in unit dosage form having between about 1.0 to about 10.0 milligrams pharmaceutical grade Na₂EDTA dihydrate dissolved in about 2.5 mL diluent or having an equimolar concentration of EDTA.

16. The pharmaceutically acceptable formulation of claim 15, wherein the solution in unit dosage has between about 2.0 to about 3.0 milligrams Na₂EDTA dihydrate dissolved in about 2.5 mL diluent or having an equimolar concentration of EDTA, and a short acting beta-2 agonist.

17. The pharmaceutically acceptable formulation of claim 16, wherein the short acting beta-2 agonist is in pharmaceutically acceptable salt form wherein the pharmaceutically acceptable salt is albuterol sulfate.

18. The pharmaceutically acceptable formulation of claim 1, wherein the pharmaceutically acceptable excipients provide an alcohol-based sanitizer, lotion or spray or a soap.

19. The pharmaceutically acceptable formulation of claim 1, wherein the pharmaceutically acceptable excipients provide an alcohol-based sanitizer containing 0.07% disodium edetate by weight.

20. The pharmaceutically acceptable formulation of claim 19, wherein the alcohol of the alcohol-based sanitizer is ethyl alcohol or isopropanol.

21. A method of treating an infection by a virus in a human subject, wherein entry by the virus into cells of the subject is by a calcium dependent pathway, wherein the composition is a pharmaceutically acceptable formulation of claim 1.

22. The method of claim 21, wherein the calcium chelating agent of the pharmaceutically acceptable formulation of claim 1 is pharmaceutical grade EDTA and wherein its pharmaceutically acceptable salt and hydrate form is sodium edetate dihydrate (Na₂EDTA·2H₂O).

23. The method of claim 22, wherein the beta 2-agonist is present in the pharmaceutically acceptable formulation.

24. The method of claim 21, wherein the virus is a single stranded RNA virus.

25. The method of claim 24, wherein the single stranded RNA virus is Rubella virus, a α-Coronavirus or a β-Coronavirus is.

26. The method of claim 24, wherein the single stranded RNA virus is a α-Coronavirus.

27. The method of claim 26, wherein the α-Coronavirus is HCoV-229E.

28. The method of claim 24, wherein the single stranded RNA virus is a β-Coronavirus.

29. The method of claim 28, wherein the β-Coronavirus is SARS-CoV-2 virus, also known as COVID-19, or SARS-CoV-1.

30. A vaccine composition, the vaccine composition comprising a live, attenuated coronavirus comprising a SARS-CoV-2 variant in which the SARS-CoV-2 fusion loops (amino acids 816-855) are replaced with the less pathogenic HCoV-229E fusion loop (amino acids 923-982) to provide substantially the same antigenicity of SARS-CoV-2 while limiting its pathogenicity to no more than the level of HCoV-229E.
